(19) United States
(12) Patent Application Publication
(10) Pub. No.: US 2006/0078484 A1
(43) Pub. Date: Apr. 13, 2006

(54) Wearable hand sanitation devices

(76) Inventor: Darcy W. Greep, South Jordan, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 11/249,547

(22) Filed: Oct. 13, 2005

Related U.S. Application Data
(60) Provisional application No. 60/618,364, filed on Oct. 13, 2004.

(51) Int. Cl. A61L 2/10; A61L 2/18

(52) U.S. Cl. 422/300; 250/454.11

(57) ABSTRACT

A wearable hand sanitizing device is structured to provide an antimicrobial agent for sanitizing the hands or other areas of the body, the device being structured to attach to the body or to an item of clothing so that the source of antimicrobial agent is readily accessible. The wearable hand sanitizing device of the present invention may be formed as a pen-like structure, as an item of jewelry or as a decal that can be worn on clothing. One embodiment of the wearable hand sanitizing device further includes a self-closing check valve to prevent dispensing of antimicrobial agent, and may include a metering device, locking device and safety features.

### CROSS-REFERENCE TO RELATED APPLICATION

This application is a non-provisional application claiming priority to provisional patent application Ser. No. 60/618,364 filed Oct. 13, 2004.

### BACKGROUND OF THE INVENTION

1. Field of the Invention

This invention relates to devices for hand-sanitation, and specifically relates to devices that are structured to be worn by a person for facilitating ready access to sanitizing preparations.

2. Description of Related Art

An increasing urban society, combined with an increasing world economy and associated travel, is bringing people into greater contact with infectious agents that can cause serious health problems, and even death. Common social practices, such as covering one's mouth when sneezing or coughing, followed by shaking hands as a greeting, also assist in the transmission of infectious organisms between people.

Viruses and bacteria passed from person to person by incidental contact in public places, such as airports, airplanes, office buildings, schools, churches, restaurants, mass transit and healthcare settings, also cause many serious infections. Nosocomial (in-hospital) infections kill thousands of people annually in the United States alone. Influenza and the common cold are readily acquired by simple contact with an infected person or a contaminated surface (such as door handles, railings, telephones, writing instruments, keyboards, money, etc.), resulting in innumerable infections annually.

Research has shown that one of the simplest and most effective means of preventing disease transmission is conscientious hand washing. However, soap, water and towels, or other hand sanitizing materials, are often not available in public places where microbial transmission and infection take place. The lack of sanitary public facilities reduces the likelihood that proper hand cleansing will be performed. In healthcare settings, hand washing is usually taught and encouraged. However, when performed improperly, or not at all, the benefits of hand washing are lost.

The inconvenience of taking time to wash properly increases the incidence of insufficient hand disinfection. Many people, particularly healthcare personnel, are reluctant to wash repeatedly due to the sheer discomfort of dry skin. Excessively dry skin with its associated cracking, increases the risk of acquiring a blood-borne infection. Lotions are often used to protect the skin of hand-washers; however, lotions themselves may be incompatible with antimicrobial soaps, rendering their activity against microbes useless. Lotion may even be a source of microbes if they reside in, or are found on, the container or its surfaces.

Most commercially available sanitizing methods include antimicrobial hand wipes, antimicrobial lotions, hand-sanitizing gels and antibacterial soaps. While successful at killing infectious microbes, access to these personal sanitizing methods are presently limited to small containers or bottles which are usually carried in a pocket or a purse. Some dispensing apparatus have been developed for wearing directly on the body. For example, U.S. Pat. No. 6,427,839 discloses a portable towelette dispenser that may be worn on a belt or other item of clothing. Hospitals are often equipped with or supply sanitizing gels or lotions for its employees, but the dispenser placements may be sparse and users may not have immediate access when needed.

It would be beneficial, therefore, to provide a device that may be worn by a user to facilitate ready access to a hand sanitizing material at any time and in any setting, which further provides a hand sanitizing material that does not produce materials that then need to be disposed of (e.g., a towelette), and which dispenses the sanitizing material by a means which does not leak or stain clothing, or is subject to misuse by children, or which results in re-contamination of the hands following use.

### BRIEF SUMMARY OF THE INVENTION

In accordance with the present invention, devices are structured for providing or dispensing hand sanitizing agent in a sanitary and simplified manner, the devices being wearable by a person to facilitate ready access to the hand sanitizing agent. The devices of the present invention may comprise many forms and embodiments, but are particularly directed to devices which repeatedly provide or dispense a sanitizing agent that is instantly usable or consumable and do not produce an immediate after-product, such as a towelette, that requires further disposal once the hand sanitizing process is complete.

The hand sanitizing devices of the present invention generally comprise a housing that is sized and dimensioned to hold or contain a hand sanitizing product in a manner that allows the user to access a quantum of the hand sanitizing agent readily upon demand. The device may preferably include a manipulable mechanism that causes the hand sanitizing agent, contained or held within the housing, to be dispensed. The hand sanitizing agent may, most suitably, be dispensed by a positive displacement of the material to prevent unnecessary waste of sanitizing material, but other means of dispensing the material are within the scope of the invention. It may also be preferred that the mechanism for dispensing the hand sanitizing agent is structured with a metering feature that dispenses a pre-determined amount of the hand sanitizing agent.

The hand sanitizing device of the present invention further includes an attachment member for securing the housing to the user's person in some fashion. The attachment member may be structured to attach the housing to the person's body, such as round the neck, wrist, arm or waist. The attachment member may also, or alternatively, be structured to attach the housing to an item of clothing, such as a pocket, belt, lapel or purse. Thus, the attachment member may be a lanyard, string, chain, clip or any other type of apparatus that can effect attachment to a body or clothing. The housing may also be attached to or configured in the form of an item of conventional jewelry, such as a watch, ring, necklace or tie tack or as a belt buckle. The hand sanitizing device may even be formed as a decal having an adhesive backing.

The hand sanitizing agent held or contained within the housing may be any suitable type or form of agent that can provide or produce an adequate microbial kill rate such that the surface with which the agent is contacted (e.g., the hands) becomes substantially free of microbes (i.e., viruses, bacteria and the like). By “substantially free of microbes” is meant that the contacted surface is sufficiently cleansed of viable microbes such that the likelihood of transmitting or infecting oneself or another person through contact with the cleaned surface is statistically low immediately following cleansing.

The hand sanitizing agent may be, for example, a gel, lotion, liquid or cream that is formulated with an antibacterial, antiviral or antimicrobial substance, or mixtures thereof. The hand sanitizing agent may also be an electronic device which, for example, emits a safe but effective amount of radio frequency or UV-light that is capable of destroying microbes without damaging the skin of the user. The hand sanitizing device may be configured to dispense a gas, such as ozone, that is effective for killing microbes. The hand sanitizing device may also be configured, in part, in the form of a cylinder, pad or any other suitable shape of absorbent material that is saturated or impregnated with a hand sanitizing liquid material and retained within a housing of the device such that when the saturated material is released or exposed from the housing, the saturated material can be contacted with the surface to be cleaned and then retracted into or covered by the housing. Many other means and methods of configuring the hand sanitizing device are possible.

### BRIEF DESCRIPTION OF THE SEVERAL VIEWS OF THE DRAWINGS

In the drawings, which illustrate what is currently considered to be the best mode for carrying out the invention;

FIG. 1 is a longitudinal cross section of a first embodiment of the invention comprising a pen-like device;

FIG. 2 is a view in cross section of the device shown in FIG. 1 taken at line 2-2;

FIG. 3 is an end view of the device shown in FIG. 1 taken at line 3-3;

FIG. 4 is a view in elevation of a second embodiment of the invention;

FIG. 5 is a view in elevation of a third embodiment of the invention;

FIG. 6 is a side view of the third embodiment shown in FIG. 5;

FIG. 7 is a view in elevation of a fourth and fifth embodiment of the invention;

FIG. 8 is a view in cross section of a sixth embodiment of the invention;

FIG. 9 is a view in cross section of a seventh embodiment of the invention; and

FIG. 10 is a view in elevation of an eighth embodiment of the invention.

### DETAILED DESCRIPTION OF THE INVENTION

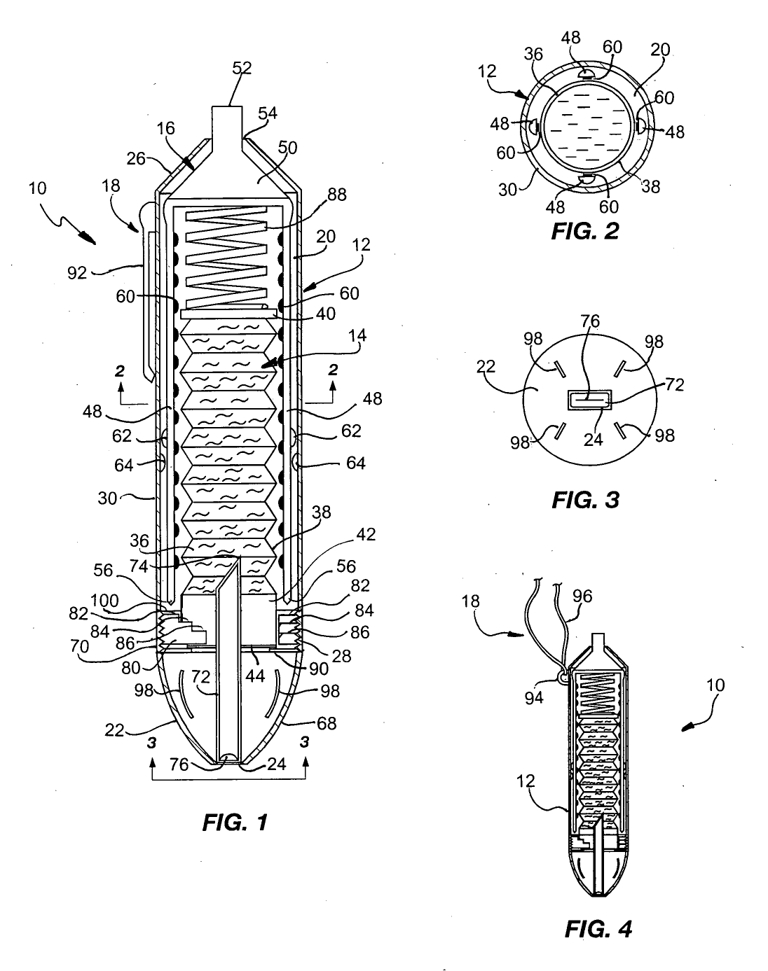

FIG. 1 illustrates a first exemplary embodiment of the present invention where the wearable hand sanitizing device 10 is configured as a pen-like element. When configured in a pen-like structure, the present invention generally comprises an elongated housing 10 that encloses a reservoir 14 that contains an antimicrobial agent, a dispensing apparatus 16 and an attachment member 18 for securing the device 10 to a person or item of clothing. The form, configuration or dimension of these basic elements may be widely variable to encompass a variety of hand sanitizing devices, and the form illustrated in FIG. 1 is but one exemplar means of carrying out the described basic elements of the invention.

In the embodiment illustrated in FIG. 1, the elongated housing 12 is generally cylindrical, having a hollow inner space 20 for receiving the reservoir 14 of antimicrobial agent. The housing 10 may have any suitable shape or dimension in cross section, and is not limited to having a circular cross section as shown in FIG. 2. The housing 12 further generally includes a tip end 22, having an opening 24 through which the antimicrobial agent is dispensed to the user, and a top end 26 at the end of the cylinder opposite the tip end 22.

Either the tip end 22 or the top end 26, or both, may be formed with screw threads 28, to be detachably attached to the main body 30 of the housing 12. FIG. 1 illustrates, for example, the tip end 22 of the housing 12 being threadedly joined to the main body 30 of the housing 12. The ability of either the tip end 2 or top end 26, or both ends, to be threadedly attachable and removable from the main body 30 of the housing 30 provides a means through which the reservoir 14 may be inserted into the housing 12, as explained more fully hereinafter. Notably, either the tip end 22 or top end 26 may, in alternative embodiments, be integrally formed with the main body 30 of the housing 30.

The reservoir 14 of the embodiment illustrated in FIG. 1 is configured as a collapsible tube 36 having a bellows-type wall 38. The collapsible tube 36 is formed at one end with a closed disk 40. The tube 36 is also formed at the opposite end with a supporting ring 42 that is not collapsible. The ring 42 is provided at its terminal axial end with a closed end wall 44 that is made of a material which can be penetrated to form an opening, as described more fully below.

The dispensing apparatus 16 illustrated in FIG. 1 comprises a plurality of rails 48 which are each connected at one end to a connecting element or spur 50 that is positioned at or in the top end 26 of the housing 12. A button 52 projects outwardly from the spur 50 and extends through an opening 54 formed in the top end 26 of the housing 12. Each rail 48 is elongated to extend substantially the length of the main body 30 of the housing 12, and each rail 48 has a free end 56.

FIG. 2 depicts that the illustrated embodiment has four rails 48. However, the dispensing apparatus 16 may have as few as two rails 48 or may have any number more than two. As further illustrated in FIG. 1, each rail 48 may be formed with a plurality of internal bosses 60 which project inwardly from the rails 48. The internal bosses 60 are evenly spaced apart down the length of each rail 48 as shown. Each rail 48 may further be formed with a least one outer boss 62 that projects in the direction of the housing 12. The housing 12 is formed with an inward projection 64 which is positioned to be aligned for contact with the outer bosses 62 of each rail 48. The inward projection 64 may be in the form of a ring that encircles the inner circumferential wall of the housing 12, or may be formed as a plurality of inwardly extending projections which are aligned with the outer bosses 62 of each rail 48.

The tip end 22 of the device 10 is illustrated as comprising a tapered portion 68 extending from a barrel portion 70 that attaches by thread means 28 to the main body 30 of the housing 12. The tapered portion 68 is formed, at its terminal end, with an opening 24. A hollow shaft 72 is connected to the opening 24 and extends along the central axis of the device 10 toward the main body 30 of the housing 12.

The inward end of the hollow shaft 72 is formed with a point 74 which is provided for puncturing the closed end wall 44 of the reservoir 14. The hollow shaft 72 thus provides a fluid communication of antimicrobial agent from the reservoir 14 to the opening 24 in the tip end 22. The hollow shaft 72 may preferably be formed with a check valve 76 at the end of the shaft 72 positioned at the opening 24, as further illustrated in FIG. 3. The check valve 76 operates such that the shaft 72 is self-closing and self-sealing after a quantum of antimicrobial agent is dispensed.

The hand sanitizing device 10 of the present invention may preferably be formed with a metering device 78 by which the amount of antimicrobial agent, when in a flowable or fluid state (including viscous materials), may be dispensed in selected amounts. FIG. 1 illustrates but one exemplar metering device 78 where the barrel portion 70 of the tip end 22 is structured with a rotatable ring 80 that, in this configuration, surrounds the supporting ring 42 of the collapsible tube 36 of the reservoir 14. A tab (not shown) which extends through a slot (not shown) in the wall of the housing 12 enables the rotational movement of the rotatable ring 80. The rotatable ring 80 is structured with more than one shoulder, illustrated in FIG. 1 as having three shoulders 82, 84, 86 that are alignable with the free ends 56 of each rail 48.

As illustrated, the hand sanitizing device 10 is assembled by removing the tip end 22, comprising the tapered portion 68 and barrel portion 70, from the main body 30 of the housing 12. The dispensing apparatus 16 is positioned in the housing 12 so that the button 52 extends through the opening 54 of the top end 26 of the housing 12, and the spur 50 is positioned in or near the top end 26. A spring 88 is inserted between the rails 48 and positioned to register against the spur 50. The reservoir 14, comprising the collapsible tube 36, is then inserted in the housing 12 between the rails 48, and the disk 40 of the collapsible tube 36 is positioned against the spring 88. The tip end 22 is then threadedly attached to the main body 30 of the housing 12.

As the tip end 22 is attached to the main body 30, the sharp tip 74 end of the shaft 72 punctures the closed end wall 44 of the collapsible tube 36. The material of the closed end wall 44 is preferably a resilient or flexible material that, once pierced by the tip 74 of the shaft 72, will conform to the circumference of the shaft 72, thereby preventing the leakage of antimicrobial agent between the punctured end wall 44 and the shaft 72. The end wall 44 of the reservoir 14 is positioned against an annular land 90 that extends inwardly from the barrel portion 70 of the tip end 22. Therefore, when the tip end 22 is attached to the main body 30 and the housing components are complete assembled, the reservoir 14 is biased between the annular land 90 and the spring 88.

In operation, when an amount of antimicrobial agent is to be dispensed, the user first determines the amount of antimicrobial agent that is to be dispensed. If the user wants a smaller portion of agent (e.g., to clean a child's hands) to be dispensed, the user rotates the rotatable ring 80 until the first or uppermost shoulders 82 of the rotatable ring 80 are axially aligned immediately below the free end 56 of each rail 48. If a slightly larger portion of agent is to be dispensed (e.g., for small adult hands), then the rotatable ring 80 is rotated so that the middle shoulder 84 is positioned in axial alignment with the free end 56 of each rail 48. If a large portion of agent is to be dispensed (e.g., for large adult hands), the rotatable ring 80 is rotated so that the lowermost shoulder 86 is axially aligned with the free end 56 of each rail 48.

Once the amount of antimicrobial agent has been selected and the rotatable ring 80 has been positioned accordingly, the button 52 of the dispensing apparatus 16 is depressed downwardly, thereby moving the rails 48 axially within the housing 12. As the outer boss 62 of each rail 48 encounters and contacts the inward projection 64 of the housing 12, each rail 48 is forced to move radially inwardly relative to the housing 12. The inward movement of the rails 48 causes the internal boss 60 of each rail 48 which is closest to the disk 40 of the collapsible tube 36 to grasp the disk 40 and force the disk 40 to move axially toward the tip end 22 of the housing 12.

Each of the rails 48 will continue to move axially toward the tip end 22 of the housing as the button 52 is depressed until the free end 56 of each rail 48 strikes the shoulder 82, 84, 86 which is axially aligned below each rail 48. When the free end 56 of each rail 48 strikes the shoulder 82, 84, 86, a selected amount of antimicrobial agent will have been forced from the reservoir 12 into the hollow shaft 72, and the pressure of the flowing agent will cause the check valve 76 to open an amount sufficient to dispense the microbial agent. Once dispensed, the check valve 76 closes again.

Upon releasing pressure from the button 52, the spring 88 forces the dispensing apparatus 16, specifically the spur 50 and rails 48, to move axially toward the top end 26 of the housing 12. Movement of the rails 48 toward the top end 26 releases the contact between the inward projection 64 and the outer boss 62 of each rail 48 so that the rails 48 move radially outwardly again and the internal bosses 60 release from the disk 40. Notably, the tension of the spring 88 is sufficient to move the dispensing apparatus 16 to a pre-depressed or starting position, but is not sufficiently tensioned to push the collapsible tube 36 in the direction of the tip end 22. The inability of the tensioned spring 88 to move the reservoir 12 axially may also be determined by the rigidity of the material from which the collapsible tube 36 is made.

The hand sanitizing device 10 of the present invention is also provided with an attachment member 18 for attaching the device 10 to a person or to the person's clothing. As illustrated in FIG. 1, the attachment member 18 may be a clip 92 of the type commonly found on pens. Alternatively, as shown in FIG. 4, the housing 12 may be configured with a ring 94 through which a string 96, chain, rope, lanyard or other similar member may be secured for suspending the hand sanitizing device 10 about a person's neck, wrist, waist, arm or other body location, or through or around an item of clothing, such as a belt loop, a button, buttonhole, purse strap or other structure.

The hand sanitizing device 10 of FIGS. 1-4 have notable features which make the device particularly convenient and safe for use. The device is, in particular, configured with a check valve 76 that prevents leakage of antimicrobial agent from the device 10. The housing 12 may also be formed with vent holes 98 in the tip end 22 of the device 10 to dissipate any negative pressure on the opening 24 and check valve 76 caused by, for example, a child sucking accidentally on the tip end 22 of the device 10.

The rotatable ring 80 may also preferably be formed with a safety shoulder 100 the height of which closely approximates or nears the free end 56 of each rail 48 such that when the rotatable ring 80 is rotated to position the safety shoulder 100 in axial alignment with the free end 56 of each rail 48, the free end 56 of each rail 48 is contacting or nearly contacting the safety shoulder, thereby preventing axial movement of the rails 48 toward the tip end 22 of the housing 12. Thus, the safety shoulder 100 provides a locking mechanism to prevent accidental dispensing of antimicrobial agent, especially if a child should gain access to the device 10.

The hand sanitizing device 10 of FIGS. 1-3 also provides a metering means as previously described by which a selected amount of antimicrobial agent may be dispensed, thereby preventing waste of antimicrobial agent. The housing 12 of the device 10 may be formed of clear plastic or have a window (not shown) formed in the main body 30 to provide the user with an indication of how much antimicrobial agent remains in the reservoir 14. The construction of the device 10 allows the reservoir 14 to be easily replaced when the antimicrobial agent is spent. The positive displacement mode of dispensing the agent also prevents unnecessary waste and allows the device 10 to be used in any direction or orientation, not just in a vertical orientation. The construction also provides dispensing of the antimicrobial agent with a single hand, and the non-contacting dispensing of the antimicrobial agent prevents cross contamination between varied users of one device.

While FIGS. 1-4 illustrate a pen-like configuration for dispensing flowable antimicrobial agents from a reservoir 16, it is within the scope of the present invention that the antimicrobial agent may be retained within the housing 12 in a reservoir 16 of sorts, but the reservoir 16 takes the form of an absorbent material that is saturated or impregnated with an antimicrobial agent. The dispensing apparatus of the device may then be structured to eject all or a portion of the absorbent reservoir from the housing to enable the user to swipe or brush the absorbent reservoir over the hands to impart the antimicrobial agent to the skin of the hands. The absorbent reservoir may then be retracted back into the housing by a retraction apparatus.

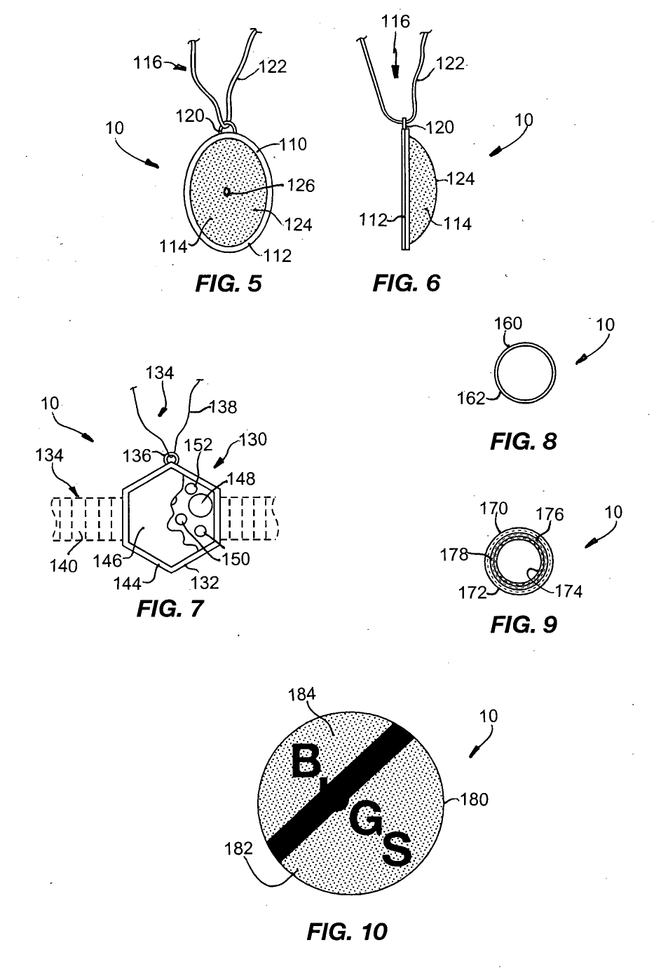

A third embodiment of the hand sanitizing device 10 of the present invention is shown in FIGS. 5 and 6, which is formed in the manner of an item of jewelry, specifically a necklace or pendant 110. The device 10 generally comprises a housing 112 which retains a reservoir 114 of antimicrobial agent. The housing 112 has an attachment member 116 shown here, by way of example, as a ring 120 through which a string 122, rope, chain, lanyard or other similar item may be positioned for attachment of the device around the user's neck, wrist, waist, arm or the like.

The reservoir 114 for retaining the antimicrobial agent may take any one of several forms. For example, the reservoir may be an absorbent material that is saturated with an antimicrobial agent so that when the reservoir 114 is rubbed over the hands, an amount of agent is released from the reservoir 114 onto the hands. Alternatively, the reservoir 114 may be a solid material that contains or is formed from an antimicrobial agent, such as triclosan, chloroxylenol or silver ions. When the solid-form reservoir 114 is abraded or contacted with the skin, a quantum of antimicrobial agent is imparted to the skin to sanitize the skin.

Still alternatively, the reservoir 114 may be a hollow form having a flexible outer wall 124 through which a minute hole 126 is formed. When an amount of antimicrobial agent is to be dispensed, pressure is applied to the flexible outer wall 126 and an amount of antimicrobial agent is thereby forced from the reservoir 114 through the minute hole 126. The size of the minute hole 126 is preferably large enough to dispense a sufficient amount of antimicrobial agent upon demand, but is sufficiently small to prevent leakage of agent from the reservoir 114. Understandably, more viscous type flowable antimicrobial agents are suitable for this embodiment. Alternatively, a minute hole (not shown) may be formed on the housing 112 and aligned with an opening in the reservoir 114 to provide dispensing of antimicrobial agent from the back side of the necklace or pendant 110. The housing 112 may be structured to releasably retain the hollow form reservoir 114 so that the reservoir 114 may be replaced when spent.

FIG. 7 illustrates a fourth embodiment of the hand sanitizing device 10 which is also structured in the manner of a wearable piece of jewelry, but the sanitizing or antimicrobial agent is provided in the form of a powered system 130. The housing 132 of this embodiment, similar to previously described embodiments, is provided with an attachment member 134 for attaching the device 10 to a wearer or item of clothing. As shown, the attachment member 134 may be a ring 136 formed on the housing 132 through which a string 138, rope, chain, lanyard or other similarly suitable member may be positioned for securing the device around the neck, wrist, waist or arm of the user, or to an item of clothing. Alternatively, as shown in phantom line, the device may be formed as a bracelet or watch-like device having a wristband 140 as the attachment member 134. The device 10 may also, or alternatively, be fitted with a clip or pin for attachment to clothing.

The housing 132 of the device 10 illustrated in FIG. 7 may generally be configured with a base member 144 and a cover 146 (shown in partial breakaway) which is attached to the base member 144. The cover 146 is preferably made of a material that is transparent or opaque to allow transmission of light through the cover 146. The sanitizing element 130 of this embodiment comprises a UV light-emitting apparatus that includes a microprocessor/battery 148 and at least one UV light source, although a plurality of light sources 150 are shown. A switch 152 is also provided for activating the microprocessor/battery 148.

In operation, the user depresses the switch 152, which activates the microprocessor/battery 148. The microprocessor/battery 148 in turn activates the UV light source(s) 150 to become illuminated. The UV light is emitted through the cover 146. The user then passes the device 10 over the area to be sanitized for a time sufficient to kill any microbes existing on the skin. The microprocessor/battery 148 is programmed to be activated for a pre-determined amount of time sufficient to assure an adequate microbial kill rate. When the UV light source(s) 150 has been activated for a sufficient amount of time to kill the microbes, the microprocessor/battery 148 automatically turns off, thereby terminating power to the UV light source(s) 150.

The device 10 illustrated in FIG. 7 is shown with a single cover 146 suitable for emitting UV light therethrough. In an alternative embodiment, the device 10 may be structured to emit UV light from both a front and back surface of the device 10. Any surface which is not light emitting, such as the sides or border of the device 10 may be treated with an antimicrobial material, such as a silver ion coating, to minimize repeat exposure to contamination when the user touches the device 10. Additionally, the powered antimicrobial agent described with respect to FIG. 7 may be employed in the pen-like structure illustrated in FIG. 1, in which case the housing 12 of the pen-like structure is made of a material that is transparent or opaque for emitting UV-light therethrough.

FIG. 8 illustrates a sixth embodiment of the hand sanitizing device 10 of the present invention where the device 10 is formed as a ring 160 wearable on a finger. The ring 160 may be made entirely of a solid antimicrobial substrate 162 containing or comprising an antimicrobial agent such as triclosan, chloroxylenol, silver ions or other commercially available antimicrobial agent. The antimicrobial substrate 162 may be abradable by contact with the area of skin to be sanitized, thereby coating the skin area with the antimicrobial agent.

Alternatively, the antimicrobial substrate 162 may be formed as an absorbent material that retains or is impregnated with an antimicrobial agent such as, for example, a gel, liquid or volatile oil, that is released upon contact with an area of skin. The device 10 may be initially sealed with a cover that, upon removal, activates the antimicrobial agent for ultimate release. The device 10 may be structured with a means, such as a color-coded band, that appears or disappears when the antimicrobial agent is spent. The ring 160 device 10 of FIG. 8 can be rubbed on the skin area to be sanitized while the ring 160 is in place on a finger, or may be removed and rubbed between the hands to release or deposit the antimicrobial agent on the hands.

FIG. 9 illustrates a seventh embodiment of the hand sanitizing device 10 that is similarly formed as a ring 170, but which is comprised of an outer ring member 172 and an inner ring member 174 that are releasably joined together to form an annular groove 176 therebetween. A middle layer 178 of antimicrobial agent is received in the annular groove 176 and is positioned to provide or release the antimicrobial agent upon contact with the area of skin to be sanitized. Release or deposition of the antimicrobial agent may occur by any of the methods previously described with respect to the embodiment shown in FIG. 8. The middle layer 178 may be replaceable by separating the outer ring member 172 from the inner ring member 174, removing the spent middle layer 178 and replacing it with a new middle layer. As previously described, the ring 170 of this embodiment may be structured with a means, such a color coded device, which indicates when the middle layer 178 is spent and requires replacement.

FIG. 10 illustrates an eighth embodiment of the hand sanitizing device 10 which is formed as a decal or sticker 180 that can be applied to a person's body or on an item of clothing. The sticker 180 generally comprises a flexible substrate 182, which may typically be made of paper, plastic or other suitable material. An antimicrobial agent is applied to a first surface 184 of the flexible substrate 182. The antimicrobial agent may be in any of the forms previously described, namely, for example, a solid material that deposits an antimicrobial agent upon abrading or contact with the skin.

Alternatively, the first surface 184 may have attached thereto a layer of absorbent material that is saturated with an antimicrobial agent, as previously described, where contact between the absorbent material and an area of skin causes a release of the antimicrobial agent onto the skin. The second surface (not seen) of the sticker 180 opposing the first surface 184 is provided with an adhesive material and a protective covering such that when the protective covering is removed to expose the adhesive material, the sticker 180 may be applied to an item of clothing, a person's body or on any other item, such as a wallet, book, etc. The sticker 180 may be printed with any desirable matter, and may include a means for indicating when the antimicrobial agent is spent.

The wearable hand sanitizing devices of the present invention are particularly structured to be worn or placed on or about a person to facilitate ready access to an antimicrobial agent for sanitizing a persons hands and/or other regions of the body. The wearable hand sanitizing devices of the present invention may be adapted for numerous types of applications, settings and uses. Therefore, reference herein to specific details of the structure or configuration of the various illustrated embodiments are by way of example only and not by way of limitation.

What is claimed is:

1. A wearable hand sanitizing device, comprising:
a housing;
a reservoir retained by said housing;
an antimicrobial agent retained by said reservoir;
a dispensing apparatus for dispensing said antimicrobial agent from said reservoir;
a self-closing valve in fluid communication with said reservoir; and
an attachment member for securing said housing to a supporting substrate on or about a person.

2. The wearable hand sanitizing device of claim 1 further comprising a metering device for selectively dispensing a determinable amount of antimicrobial agent from said reservoir.

3. The wearable hand sanitizing device of claim 1 further comprising a locking mechanism for preventing the accidental dispensing of said antimicrobial agent from said housing.

4. The wearable hand sanitizing device of claim 1 further comprising safety vents formed in said housing to prevent unintended dispensing of antimicrobial agent from said self-closing valve under application of negative pressure.

5. The wearable hand sanitizing device of claim 1 wherein said housing is configured in the form of a pen-like structure having an internal space for receiving said reservoir.

6. The wearable hand sanitizing device of claim 5 wherein said reservoir is a collapsible tube containing said antimicrobial agent.

7. The wearable hand sanitizing device of claim 5 wherein said reservoir is formed of an absorbent material impregnated with said antimicrobial agent which is releasable from said reservoir upon contacting said reservoir with a surface to be cleaned.

8. The wearable hand sanitizing device of claim 1 wherein said attachment member is a clip secured to housing.

9. The wearable hand sanitizing device of claim 1 wherein said attachment member further comprises a ring for receiving a lanyard or elongated string-like member.

10. The wearable hand sanitizing device of claim 1 wherein said antimicrobial agent is a flowable fluid.

11. The wearable hand sanitizing device of claim 1 wherein said antimicrobial agent is a viscous material.

12. A wearable hand sanitizing device, comprising:
a housing;
an attachment member secured to said housing providing means for securement of said housing to a support surface on or about a person; and
an antimicrobial agent in the form of a UV-light source.

13. The wearable hand sanitizing device of claim 12 wherein said housing is in the form of a pen-like structure having a hollow space for retaining said UV-light source and having a light-emitting wall for emitting UV-light through said wall.

14. The wearable hand sanitizing device of claim 12 wherein said housing is in the form of an item of jewelry for wearing on or about a person.

15. The wearable hand sanitizing device of claim 12 wherein said housing includes at least one surface to which is applied an adhesive material for securing said housing to a support surface on or about a person.

16. A wearable hand sanitizing device, comprising:
a housing configured in the form of an item of jewelry for wearing on a person's body;
a reservoir attached to said housing; and
a dispensible antimicrobial agent retained in said reservoir.

17. The wearable hand sanitizing device of claim 16 wherein said housing is configured as a necklace, pendant, pin, ring, bracelet, watch-like member or belt buckle.

18. The wearable hand sanitizing device of claim 17 wherein said reservoir is a hollow structure configured to retain a flowable antimicrobial agent.

19. The wearable hand sanitizing device of claim 17 wherein said reservoir is constructed of an absorbent material impregnated with an antimicrobial agent that is dispensible upon contacting said reservoir with an area of skin to be sanitized.

20. The wearable hand sanitizing device of claim 17 wherein said reservoir is constructed as a solid form made from an antimicrobial agent which, upon abrading the solid form, deposits an amount of antimicrobial agent on the area contacted for sanitizing.
